(19) United States
(12) Patent Application Publication
(10) Pub. No.: US 2021/0093714 A1
(43) Pub. Date: Apr. 1, 2021

(54) Combination and method for inhibiting the cancer cells

(71) Applicant: NATIONAL TAIWAN UNIVERSITY, Taipei (TW)

(72) Inventors: Cheng-Chi Chang, Taipei City (TW); Yue-Ju Li, Taipei City (TW)

(73) Assignee: NATIONAL TAIWAN UNIVERSITY, Taipei (TW)

(21) Appl. No.: 14/088,326

(22) Filed: Nov. 22, 2013

(51) Int. Cl. A61K 39/395; A61K 38/39; A61K 31/337; A61K 31/704; A61K 31/5377; A61P 35/00

(52) U.S. Cl. CPC A61K 39/3955; A61K 38/39; A61P 35/00; A61K 31/704; A61K 31/5377; A61K 31/337

(57) ABSTRACT

The present invention provides a combination for inhibiting the cancer cells, which comprises a pharmaceutically effective dose of CCN2 or a CCN2 functional fragment, and a pharmaceutically effective dose of a cancer drug; the present invention also provides a method for inhibiting the cancer cells, which comprises administrating to the cancer cells a pharmaceutically effective dose of CCN2 or a CCN2 functional fragment, and a pharmaceutically effective dose of a cancer drug.

### CROSS-REFERENCE TO RELATED APPLICATIONS

This application claims the benefit of C-C Chang et al., Cell Death and Differentiation, 2013, 20, p.443-p.455, published on Nov. 23, 2012, the contents of which are incorporated by reference.

### BACKGROUND OF THE INVENTION

### Field of the Invention

The present invention relates to a combination and method for inhibiting the cancer cells, and more particularly relates to a pharmaceutical combination and method for inhibiting the cancer cells, which comprises CCN2 or CCN2 functional fragment, wherein CCN2 or CCN2 functional fragment can activate anoikis by activating EGFR degradation to inhibit cancer cells.

### Description of the Related Art

Cancer cells can activate specific growth factor receptor, which includes epidermal growth factor receptor (abbreviated as EGFR). When EGFR is activated, its downstream signal pathway is also activated to inhibit anoikis and results in the metastasis of cancer cells. Anoikis can prevent the cancer cells which are detached from the extracellular matrix (abbreviated as ECM) from becoming metastatic (Benvenuti S, Comoglio P M., J Cell Physiol, 2007, 213, 316-325.; Geiger T R, Peeper D S., Cancer Res, 2007, 67, 6221-6229. ; Reginato M J, Mills K R, Paulus J K, Lynch D K, Sgroi D C, Debnath J et al., Nat Cell Biol, 2003, 5, 733-740). The relationship between EGFR and anoikis still has not been clarified.

Connective tissue growth factor (abbreviated as CCN2) is a member of CCN family of secreted, matrix-associated proteins that play various roles in angiogenesis and tumor growth. CCN2 can interact with a variety of extracellular molecules, thereby regulating various cellular functions such as chemotaxis, invasion, and metastasis.

CCN2 is highly expressed in normal tissues or pre-cancer lesions in colorectal cancer (Chang C C, Lin B R, Che T F, Chen S T, Chen Robert J C, Yang C Y, Jeng Y M, Liang J T, Lee P H, Chang K J, Chau Y P, Kuo M L, Gastroenterolgy., 2005, 128, 9-23) and lung adenocarcinoma (Chang C C, Shih J Y, Jeng Y M, Su J L, Lin B Z, Chen S T, Chau Y P, Yang P C, Kuo M L, J Natl Cancer Inst., 2004, 96, 364-75). It was also found that the expression of CCN2 is progressively declining when cancer cells of many cancers, such as colorectal cancer, lung adenocarcinoma, and oral squamous cell carcinoma, begin to metastasize (Chu C Y, Chang C C, Prakash E, Kuo M L, J Biomed Sci., 2008, 675-685). For lung cancer and colorectal cancer patients, a decrease inexpression of CCN2 was significantly correlated with a decrease in survival of patients. In addition, a increase in expression of CCN2 in lung adenocarcinoma cells of experimental animals can suppress the metastasis of the lung adenocarcinoma cells. The evidence indicates that CCN2 can inhibit metastasis of lung adenocarcinoma cells, but its signal pathway has not been clarified.

So far, lung cancer is still a common cause of cancer deaths in the world. In recent years, the treatment of lung cancer has been improved, butthe survival rate of patients with lung cancer is still quite low. Patients with lung cancer has high mortality rate because most lung cancers begin to grow silently, without any symptoms. The majority of lung cancer, especially non-small cell lung cancer (abbreviated as NSCLS), is often diagnosed at a relatively late stage and the cancer cells have metastasized. NSCLS can be divided into three subtypes: adenocarcinoma, squamous-cell carcinoma and large-cell carcinoma; adenocarcinoma (when the cancer appears in lung, it also called lung adenocarcinoma) is the most common type.

The current lung cancer treatments can be divided into chemotherapy and targeted therapy. Chemotherapy drugs include paclitaxel and doxorubicin as active pharmaceutical ingredients. paclitaxel has been approved to treat breast cancer, NSCLS and pancreatic cancer. doxorubicin has been approved to treat ovarian cancer, breast cancer, NSCLS and AIDS-related Kaposi's sarcoma.

Targeted therapy drugs include gefitinib, erlotinib and cetuximab as active pharmaceutical ingredients, which target EGFR. Gefitinib and erlotinib are small molecule drugs that can inhibit EGFR tyrosine kinase activity. Gefitinib has been approved to treat NSCLS. Erlotinib has been approved to treat NSCLS and pancreatic cancer. Cetuximab is macromolecular drug (monoclonal antibody) which can target EGFR to inhibit EGFR overexpression in lung adenocarcinoma cells. Cetuximab has been approved to treat head and neck cancer and colorectal cancer.

Drug resistance which often occurs in curing a disease or condition reduces the effectiveness of a drug. It has to be overcome with higher drug dose. For example, EGFR mutation wihch may occur in some cancer patients causes resistance to EGFR targeted drugs and reduces the sensitivity to cancer drugs. Chemotherapy drugs often have serious side effects. Therefore, how to overcome drug resistance to enhance the sensitivity to cancer drugs is an urgent problem.

There is clearly a need in the art for improving the effectiveness of cancer treatment or reducing the dose of cancer drugs , the present invention can clarify the role of CCN2 in cancer treatment, and CCN2 can be used as chemotherapy and targeted therapy auxiliary ingredient to effectively solve the problem of drug resistance.

### BRIEF SUMMARY OF THE INVENTION

In view of the above objective, the present invention provides a combination for inhibiting the cancer cells, comprising a pharmaceutically effective dose of CCN2 or a CCN2 functional fragment, and a pharmaceutically effective dose of a cancer drug.

In one embodiment, the CCN2 functional fragment comprises a CT domain.

In one embodiment, the CCN2 functional fragment comprises cysteines in positions 273 and 287, counted from the N-terminus.

In one embodiment, CCN2 or the CCN2 functional fragment has a concentration between 5 ng/mL and 200 ng/mL. In one preferred embodiment, the concentration thereof is about 100 ng/mL.

In one embodiment, the cancer drug is a cancer chemotherapy drug.

In one embodiment, the cancer chemotherapy drug is paclitaxel or doxorubicin.

In one embodiment, paclitaxel has a concentration between 1 μM and 10 μM. In one preferred embodiment, the concentration thereof is about 10 μM.

In one embodiment, doxorubicin has a concentration between 0.5 μM and 10 μM. In one preferred embodiment, the concentration thereof is about 5 μM.

In one embodiment, the cancer drug is an EGFR targeted drug.

In one embodiment, the EGFR targeted drug is an EGFR tyrosine kinase inhibitor or an EGFR monoclonal antibody.

In one embodiment, the EGFR tyrosine kinase inhibitor is gefitinib.

In one embodiment, the EGFR monoclonal antibody is cetuximab.

In one embodiment, the cancer cells are non-small cell lung cancer cells, large intestine cancer cells, colorectal cancer cells, breast cancer cells, pancreatic cancer, ovarian cancer, AIDS-related Kaposi's sarcoma cells, head and neck cancer, squamous cell carcinoma, colon cancer cells, skin cancer cells, prostate cancer cells, cervical cancer cells, gastric cancer cells, esophageal cancer cells, brain cancer cells or bladder cancer cells.

Further, the present invention also provides a method for inhibiting the cancer cells, comprising administrating to the cancer cells a pharmaceutically effective dose of CCN2 or a CCN2 functional fragment, and a pharmaceutically effective dose of a cancer drug.

In one embodiment, the CCN2 functional fragment comprises a CT domain.

In one embodiment, the CCN2 functional fragment comprises cysteines in positions 273 and 287, counted from the N-terminus.

In one embodiment, the cancer drug is a cancer chemotherapy drug.

In one embodiment, the aforementioned cancer chemotherapy drug is paclitaxel or doxorubicin.

In one embodiment, the aforementioned cancer drug is an EGFR targeted drug.

In one embodiment, the aforementioned EGFR targeted drug is an EGFR tyrosine kinase inhibitor or an EGFR monoclonal antibody.

In one embodiment, the aforementioned EGFR tyrosine kinase inhibitor is gefitinib.

In one embodiment, the aforementioned EGFR monoclonal antibody is cetuximab.

In one embodiment, the aforementioned cancer cells are non-small cell lung cancer cells, large intestine cancer cells, colorectal cancer cells, breast cancer cells, pancreatic cancer, ovarian cancer, AIDS-related Kaposi's sarcoma cells, head and neck cancer, squamous cell carcinoma, colon cancer cells, skin cancer cells, prostate cancer cells, cervical cancer cells, gastric cancer cells, esophageal cancer cells, brain cancer cells or bladder cancer cells.

### BRIEF DESCRIPTION OF THE DRAWINGS

FIG. 1 is a schematic diagram of the present invention showing the mechanism of CCN2-induced metastatic suppression of lung adenocarcinoma cells;

FIG. 2A shows a western blot analysis of the expression of CCN2 in the lung adenocarcinoma cell lines, CL1-5 and A549, transduced with CCN2 and in the lung adenocarcinoma cell line, CL1-0, transduced with siCCN2;

FIG. 2B shows the membranous association between EGFR and CCN2 in lung adenocarcinoma cell lines, CL1-5 and A549;

FIG. 2C shows the physical association between EGFR and CCN2 in lung adenocarcinoma cell line, A549;

FIG. 2D shows the effect of TrkA on the physical association between CCN2 and EGFR;

FIG. 2E shows the effect of Erbitux, an EGFR monoclonal antibody, on the physical association between CCN2 and EGFR in the lung adenocarcinoma cell line, A549;

FIG. 2F shows the sequential CT-domain deletion constructs or CT-domain construct of CCN2;

FIG. 2G shows the effect of EGF, an EGFR ligand, and Erbitux, an EGFR monoclonal antibody, on the physical association between a CT domain of CCN2 and EGFR;

FIG. 2H shows the effect of the mutations of the cysteine residues, C273A and C287A, of the CT domain of CCN2 on the association between EGFR and CCN2 or between EGFR and Src;

FIG. 3A shows the effect of rCCN2 on the phosphorylation of ERK in the lung adenocarcinoma cell lines, CL1-5, A549 and CL1-0;

FIG. 3B shows the effect of CCN2 on the phosphorylation of ERK in attached lung adenocarcinoma cell lines, CL1-5 and A549, and in the suspended lung adenocarcinoma cell lines, CL1-5 and A549;

FIG. 3C shows the effect of CCN2 on the formation of EGFR/Src complex in the lung adenocarcinoma cell lines, CL1-5 and A549;

FIG. 3D is the effect of CCN2 on the phosphorylation of Src in the lung adenocarcinoma cell lines, CL1-5 and A549;

FIG. 3E is the effect of rCCN2 on the phosphorylation of Src in lung adenocarcinoma cell line, CL1-5;

FIG. 4A shows the effect of CCN2 on the degradation of EGFR in the lung adenocarcinoma cell line, CL1-5, which was treated with MG132;

FIG. 4B shows the effect of CCN2 on the degradation of EGFR in the lung adenocarcinoma cell line, CL1-5, which was treated with cycloheximide;

FIG. 4C shows the effect of rCCN2 on the ubiquitination degree of EGFR in the lung adenocarcinoma cell line, CL1-5;

FIG. 4D shows the effect of rCCN2 on the formation of EGFR/c-Cbl complex in the lung adenocarcinoma cell lines, CL1-5;

FIG. 4E shows the effect of CCN2 on the formation of EGFR/c-Cbl complex in the lung adenocarcinoma cell lines, CL1-5 and A549;

FIG. 4F shows the effect of CCN2 on the formation of P-pix/c-C131 complex and the phosphorylation of β-pix in the lung adenocarcinoma cell lines, CL1-5, A549 and CL1-0;

FIG. 4G shows the effect of rCCN2 on the formation of P-pix/c-Cbl complex and the phosphorylation of β-pix in the lung adenocarcinoma cell line, CL1-5;

FIG. 4H shows the effect of v-Src and rCCN2 on the formation of P-pix/c-Cbl complex, the phosphorylation of β-pix and the degradation of EGFR in the lung adenocarcinoma cell line, CL1-5, and the effect of the Src^{Y4I6F} mutant on the formation of β-pix/c-Cbl complex, β-pix phosphorylation and EGFR degradation in the lung adenocarcinoma cell line, CL1-0;

FIG. 4I shows the effect of the P-pix^{Y422F} mutant on the formation of p-pix/c-Cbl complex, EGFR degradation and DAPK expression in the lung adenocarcinoma cell line, CL1-0;

FIG. 5A shows the effect of different concentrations of rCCN2 on DNA fragments in the attached or the suspended lung adenocarcinoma cell lines, CL1-5;

FIG. 5B shows the effect of CCN2 on DNA fragments in the suspended lung adenocarcinoma cell lines, CL1-5, A549 and CL1-0;

FIG. 5C shows the effect of different concentrations of anti-CCN2 on DNA fragments in the suspended lung adenocarcinoma cell line, CL1-5;

FIG. 5D shows the effect of different CCN2 mutants, Src and β-pix on anoikis in the suspended lung adenocarcinoma cell line, CL1-5;

FIG. 5E shows the effect of a Src^{Y416F} mutant and a β-piX^{Y422F} mutant on anoikis in the suspended lung adenocarcinoma cell line, CL1-5;

FIG. 5F shows the effect of rCCN2 on anoikis in the lung adenocarcinoma cell line(in vivo), CL 1-5;

FIG. 5G shows the effect of rCCN2 on the change of metastasis of the lung adenocarcinoma cell lines(in vivo), CL1-5;

FIG. 6A shows the effect of the CT domain of CCN2 on the expression of DAPK in the lung adenocarcinoma cell line, CL1-5;

FIG. 6B shows the effect of rCCN2 on the expression of DAPK at different times in thelung adenocarcinoma cell lines, CL1-5, A549 and CL1-0;

FIG. 6C shows the effect of rCCN2 on the expression of DAPK under different doses and DNA fragments in the lung adenocarcinoma cell lines, CL1-5, A549 and CL1-0;

FIG. 6D shows the effect of MEK-1 and CCN2 on the expression of DAPK and anoikis in the lung adenocarcinoma cell line, CL1-5;

FIG. 6E shows the effect of PD98059 and CCN2 on the expression of DAPK and anoikis in the lung adenocarcinoma cell line, CL1-5;

FIG. 6F and FIG. 6G show the effect of CCN2 and DAPK on the number of metastatic lung nodules, weight of lung and survival time of cells in the lung adenocarcinoma cell line(in vivo), CL1-5;

FIG. 6H shows the effect of CCN2 and DAPK on Lung adenocarcinoma patients;

FIG. 7 shows the effect of rCCN2 on the expression of wild-type and mutated-type EGFR;

FIG. 8A shows the effect of different concentrations of chemotherapeutic drugs and CCN2 on the rate of cell apoptosis in the lung adenocarcinoma cell line, CL1-5;

FIG. 8B shows the effect of different concentrations of the EGFR tyrosine kinase inhibitor, gefitinib, on the lung adenocarcinoma cell line, PC9/WT, and the drug resistant lung adenocarcinoma cell line, PC9/IR;

FIG. 8C shows the effect of rCCN2 on the lung adenocarcinoma cell line, PC9/WT, and the drug resistant lung adenocarcinoma cell line, PC9/IR;

FIG. 8D and FIG. 8E show the effect of rCCN2 and EGFR tyrosine kinase inhibitor, gefitinib, on attached and suspended drug resistant lung adenocarcinoma cell line, PC9/IR; and

FIG. 8F shows the effect of the EGFR monoclonal antibody, cetuximab (brand name Erbitux), and different concentrations of rCCN2 on the rate of cell survival in the attached lung adenocarcinoma cell line, CL1-5.

### DETAILED DESCRIPTION OF THE INVENTION

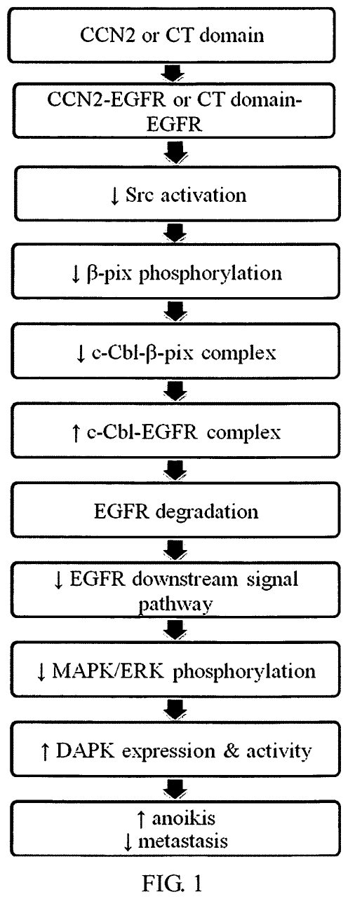

The present invention provides a composition and method for inhibiting the cancer cells, which comprises a pharmaceutical effective dose of CCN2 or CT domain of CCN2, and a pharmaceutical effective dose of a cancer drug. The experiments show that CCN2 or a CT domain of CCN2 used in the present invention can inhibit the metastasis of lung adenocarcinoma cell lines through the mechanism shown in FIG. 1. In lung adenocarcinoma cell lines, CCN2 binds to EGFR through the CT domain, thus inhibiting thephosphorylation of Src. Then, by blocking the formation of β-pix/c-Cbl(c-casitas B-lineage lymphoma) complex and promoting the formation of EGFR/c-Cbl complex, the degradation of EGFR is induced to inhibit the downstream signal pathway of EGFR. Then, by inhibiting the phosphorylation of ERK/MAPK, the expression and activity of DAPK can be activated to promote anoikis and to inhibit the metastasis of lung adenocarcinoma cell lines.

Detailed embodiments of the present invention will be described in the following contents with reference to the drawings, It is noted that the following embodiments are based on lung adenocarcinoma cell lines as an example, but the present invention is not limited to this type of cancer cell lines. A brief description of materials and methods of the present invention is shown below.

### Materials and Methods

### Cell Culture

The preferred embodiments of the invention use lung adenocarcinoma cell lines. Lung adenocarcinoma cells were grown in RPMI-1640 medium with 10% fetal bovine serum bbreviated as FBS) and 2 mM L-glutamine at 37° C. in a humidified atmosphere of 5% CO₂-95% air. CL1-0 was the parent cell line. CL1-5 was selected from CL1-0 cultures with a polycarbonate membrane coated with Matrigel in a Transwell invasion chamber as described previously. A549 cells were obtained from the American Type Culture Collection. Adherent cells were detached from the culture dishes with trypsin-EDTA.

### Construction of CCN2-Expression Plasmids

Total RNA was extracted from CL1-0 cells, and cDNA of CCN2 was cloned and amplified by RT-PCR with the primers 5′-ATGACCGCCGCCAGTATGG-3′(SEQ ID NO: 1) and 5′-TCATGCCATGTCTCCGTACATCTT-3′ (SEQ ID NO: 2, PubMed serial number: XM-037056). The X was then subcloned into a pcDNA3N5-His TOPO TA vector (Invitrogen Corporation, San Diego, Calif.).

### Construction of CCN2-Deletion Mutants

Serial deletion mutants of CCN2 were generated by deleting the CT domain, CT and TSP-1 domains, and CT, TSP-1, and VWC domains; designated as CCN2/d3, CCN2/d2, or CCN2/d1, respectively. The deletion constructs were generated using the reverse primer 5′-CGGAATTCAACCATGACCGCCGCCAGT-3¹(SEQ ID NO: 3) in combination with the forward primers: 5′-GCTCTAGATCAGATGCACTTTTTGCCCTTC-3′(SEQ ID NO: 4); 5′-GCTCTAGATCAGTCTGGGCCAAACGTGTCT-3′(SEQ ID NO: 5); and 5′-GCTCTAGATCAGCAGGAGCACCATCTTTG-3′(SEQ ID NO: 6), respectively.

CCN2 siRNA Constructs

Candidate siRNA oligos were targeted against the 379-bp region at the 30 end of exon 2 of murine CCN2. The siRNA oligos were synthesized and cloned into the pSilencer 1.0 vector under control of the U6 RNA Polymerase III promoter, according to the manufacturer's instructions (Ambion, Austin, TX, USA). Of the two siRNA constructs tested, the greatest knockdown was seen with the oligo targeting CCN2379-397 (5′-CCTATTCTGTCACTTCGGC-3′, SEQ ID NO: 7). The ability to silence CCN2 was tested by transient cotransfection with expression vectors encoding murine CCN2 into human 293 cells ans cells of the human lung adenocarcinoma cell lines A549 and CL1-5.

### Plasmid and Transient Transfection

The deletion constructs, point mutation expression plasmids, and vectors were transiently transfected into CL1-5 and A549 cells using the TransFast transfection reagent (Promega Corporation). We used 3 mg plasmids and 8 mg transfection reagents, according to the manufacturer's instructions. One hour after transfection, the cells were placed in normal complete medium and cultured for a further 8 h. The transfected cells were harvested and subjected to invasion assay, promoter assay, and western blot analysis.

### Stable Transfected Clone Selection

Purified plasmid DNA (3 mg) was transfected into XL1-5 and A549 cells, or silence RNA plasmid (3 mg) of CCN2 was transfected into CL1-0 cells, using the TransFast transfection reagent (Promega Corporation). Twenty-four hours after transfection, gentamicin (G418; Life Technologies Corporation, Carlsbad, Calif., USA) was added to 600 mg/ml for stable transfectant selection. Thereafter, the selection media was replaced every 3 days. After 2 week of selection in G418, clones of resistant cells were isolated and allowed to proliferate in 100 mg/ml G418-containing medium. Integration of transfected plasmid DNA was confirmed by RT-PCR ansd western blot analysis.

Western Blot Analysis

Proteins (40 mg) were separated by SDS-PAGE and transferred to a polyvinylidene difluoride membrane (Immobilon-P membrane; Millipore) by electrotransfer. The blot was blocked in a solution of 5% skim milk and 0.1% Tween 20 in PBS. Thereafter, membrane-bound proteins were probed with the following primary antibodies: CCN2, DAPK, phosphorylated ERK, ERK-1, phosphorylated Akt, Akt, TrkA, phosphorylated-Src, Src, b-actin, a-tubulin (Santa Cruz Biotechnology), p-EGFR, and EGFR (Cell Signaling Technology, Danvers, MA, USA). The membrane was washed and then incubated with HRP-conjugated secondary antibodies for 30 min. Enhanced chemiluminescence reagents (Amersham Pharmacia Biotech, Piscataway, N.J., USA) were employed to depict the protein bands on membranes. The light was captured on Kodak X-Omat Blue autoradiography film (PerkinElmer Life Sciences, Boston, Mass., USA).

### Immunoprecipitation

CCN2 transfectants were seeded overnight and incubated in serum-free media for 24 h. For immunoprecipitations, cells were lysed in an RIPA buffer (150 mM NaCl, 50 mM Tris-base, 5 mM EDTA, 1% NP-40, and 0.25% deoxycholate; pH 7.4). Protein concentrations were determined using the BCA protein assay kit (Pierce, Rockford, Ill., USA). Lysate was incubated for 2 h at 4□ with gentle rotation with rabbit polyclonal antibodies for human EGFR, CCN2, or Src immobilized onto protein A—sepharose beads (Santa Cruz Biotechnology, Santa Cruz, Calif., USA). Beads were washed twice with an IP buffer (137 mM NaCl, 2.7 mM KCl, 1 mM MgCl₂, 1 mM CaCl₂, 1% NP-40, 10% glycerol, 1 mg/ml bovine serum albumin, and 20 mM Tris; pH 8.0), boiled in a sodium dodecyl sulfate-polyacrylamide gel electrophoresis (SDS-PAGE) sample buffer, and centrifuged (12000×g, 4° C. for 15 min).

### In Vitro Binding Assay

The recombinant proteins rCCN2 (BioVendor Com. Heidelberg, Germany), rEGFR, and rEGF (R&D Techne Corporation) (2 mg) were added in binding buffer (50 mM sodium phosphate, pH 7.5, 500 mM NaCl, and I% Nonidet P-40) and incubated with gentle rotation at 4° C. for 2 h. Thereafter, crosslinks were reversed by boiling in an SDS-PAGE sample buffer (containing 0.1M dithiothreitol (DTT)) before loading on SDS-PAGE gels for immunoblotting analysis. To map EGFR and CCN2 interactions, CCN2 and EGFR antibodies (Cell Signaling Technology) were used to detect the specific bands.

### Fluorescence-Activated Cell Sorting(FACS)

Cells at 80% confluent growth were placed in fresh serum-free medium for 16 h; thereafter, they were treated with 100 ng/ml rCCN2 (BioVendor Com.) or 20 ng/ml rEGF (R&D Techne Corporation) for 15 min. Cells were resuspended and incubated with primary polyclonal antibody against CCN2 or EGF (Santa Cruz Biotechnology) for 1 h at 4° C. After washing, cells were stained with an FITC-labelled goat anti-rabbit secondary antibody (Santa Cruz Biotechnology) for 30 min in the dark. Analysis was then conducted with an FACS Calibur cytometer (Collaborative Biomedical, BD Biosciences).

### Anoikis Assay

Cells were placed in growth medium in suspension at a density of 500 000 cells per ml. They were then either placed on ultralow-binding hydrogel-coated 35-mm culture plates (Coming Incorporated Life Sciences, Lowell, Ma., USA) for 24 h. Condition media was collected and used to determine apoptosis with the cell-death detection ELISA kit (Roche Corporation, Boulder, Colo., USA) according to the manufacturer's instructions. Each point was performed in duplicate and each experiment was conducted at least six times.

High-Frequency Ultrasonic Imaging

Mice were anesthetized with isoflurane inhalation, and biofluorescence images were photographed with a CCD camera (IVIS; Xenogen Corporation, Caliper Life Sciences, Hopkinton, Mass., USA) at specific times. Imaging times ranged from 1 to 60 s, depending on the amount of fluorescence activity. Biofluorescence from the region of interest (ROI) was defined manually, and the data were expressed as photon flux (photons/s/cm²/steradian). Background photon flux was defined using an ROI from a mouse that was not given an i.p. injection of cancer cells. All biofluorescence data were collected and analyzed using IVIS. Celltracker dye was purchased from Molecular Probes, Invitrogen Corporation.

Reverse Transcription Polymerase Chain Reaction(RT-PCR)

Reverse transcription of RNA isolated from cells was performed in a final reaction containing the following: total RNA (5 mg), First Strand Buffer with DTT (10 mM), deoxy ribonucleotide triphosphate (dNTP) (2.5 mM), Oligo (dT) 12-18 primer (1 mg), and 200 U/I Moloney murine leukemia virus reverse transcriptase (200 U). The reaction was conducted at 37° C. for 2 h and was terminated by heating the solution to 70° C. for 10 min. Thereafter, 1 I of the reaction mixture was amplified by PCR using the following pairs of primers: 5′-CCAGCAGCAGGCAGCACTTG-3′ (sense) (SEQ ID NO: 8) and 5′-CACGGGCGCTGCACCACTAC-3′ (antisense) (SEQ ID NO: 9), to produce a 420-bp fragment of the DAPK gene. The internal control gene was b-actin, for which we used 5′-GATGATGATATCGCCGCGCT-3′ (sense) (SEQ ID NO: 10) and 5′-TGGGTCATCTTCTCGCGGTT-3′ (antisense) (SEQ ID NO: 11) to produce a 320-bp fragment product. A 220-bp fragment of the Bc1-2 gene was produced by 5′-TGTGGCCTTCTTTGAGTTCG-3′ (sense) (SEQ ID NO: 12) and 5′-AGCAGAGTCTTCAGAGACAG-3′ (antisense) (SEQ ID NO: 13). The primers 5′-TGGTATCGTGGAAGGACTCATGAC-3′ (sense) (SEQ ID NO: 14) and 5′-ATGCCAGTGAGCTTCCCGTTCAGC-3′ (antisense) (SEQ ID NO: 15) produced a 189-bp fragment product of the internal control gene GAPDH. PCR amplification was conducted in a reaction buffer containing 20 mM Tris-HCl (pH 8.4), 50 mM KCl, 1.5 mM MgCl₂, 167 mm, dNTPs, 2.5 U of Taq DNA polymerase, and 0.1 mm primers. The reactions were performed in a Biometra Thermaoblock (Biometra Inc, Baltimore, MD, USA) using the following program: denaturing for 1 min at 95° C., annealing for 1 min at 58° C., and elongating for 1 min at 72° C., 23 cycles in total; the final extension occurred at 72° C. for 10 min. Equal volumes of each PCR sample were subjected to electrophoresis on a 1% agarose gel, which was then stained with ethidium bromide and photographed under UV illumination.

### Dual Luciferase Reporter Assay(Promoter Activity Assay)

For cell transfection, cells were seeded in six-well plates. After reaching approximately 70% confluence, cells were transfected with PGL2-basic and DAPK full-length promoter using TransFast (Promega Corporation). After transfection, the medium was replaced by fresh normal growth medium, and the cells were incubated for 24 h. After starvation in serum-free medium for 16 h, the cells were harvested. The luciferase activity was determined using a dual-luciferase reporter assay system (Promega Corporation) and was measured with a luminometer.

### Kaplan-Meier Survival Plots

Survival curves using the Kaplan-Meier method, by log-rank test, testing recurrence and survival time differences in cancer patients with high expression of CTGF and low expression of CTGF. And calculate the median survival time of the survival curves and its 95% confidence interval.

### Immunohistochemistry

Tissue sections for immunostaining were obtained from formalin-fixed and paraffin-embedded primary tumors produced in mice by tail vein-injected human lung adenocarcinoma cell lines. Sections were performed with anti-human DAPK, CCN2, and EGFR antibodies (Santa Cruz Biotechnology). After three washes in PBS, the samples were treated with biotin-labeled secondary antibodies (Vector Laboratories, Burlington, ON, USA) at a dilution of 1:500 for 1 h at room temperature. Bound antibodies were detected with an ABC kit (Vector Laboratories). The slides were stained with diaminobenzidine, washed, counterstained with Delafield's hematoxylin, dehydrated, treated with xylene, and mounted.

In Vivo Experimental Metastasis

Cells were washed and resuspended in PBS. Subsequently, 6-week-old SCID mice were injected in the lateral tail vein with a single-cell suspension containing 106 cells in 0.1 ml PBS. The mice were injected i.v. with cells so that we could measure the metastatic potential and perform biofluorescence imaging. The mice were killed after 8 weeks, and all of their organs were examined for metastasis formation. The lungs were removed and fixed in 10% formalin fixative. The number of lung-tumor colonies was counted under a dissecting microscope.

### Statistics

Summary statistics are presented as the mean±S.D. Where appropriate, data were evaluated by performing a simple comparison between two values using Student's t-test and the Tukey's Studentized Range (HSD) Test. All statistical analyses were performed with the SPSS program (version 10.0). Variables were retained in a model if the associated two-tailed P-values were ≤0.10 (all statistical tests were two-tailed). A P-value of <0.05 was considered statistically significant.

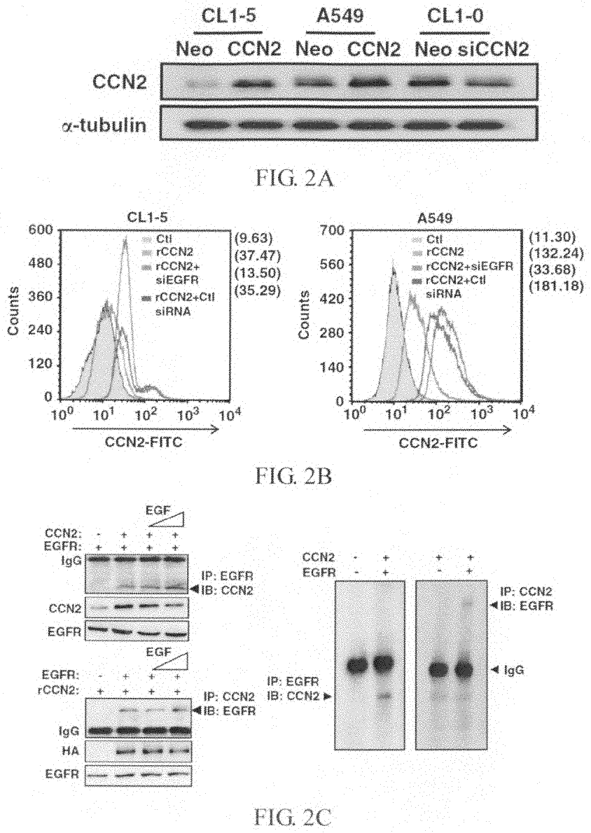

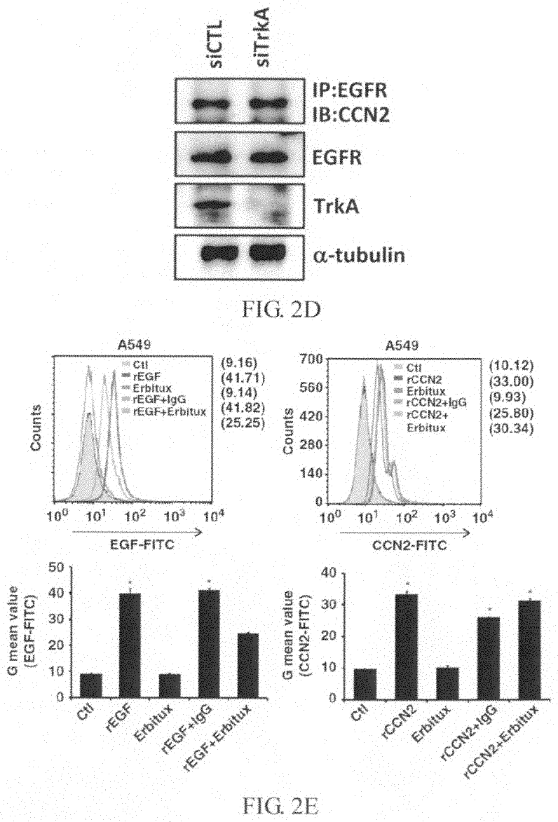

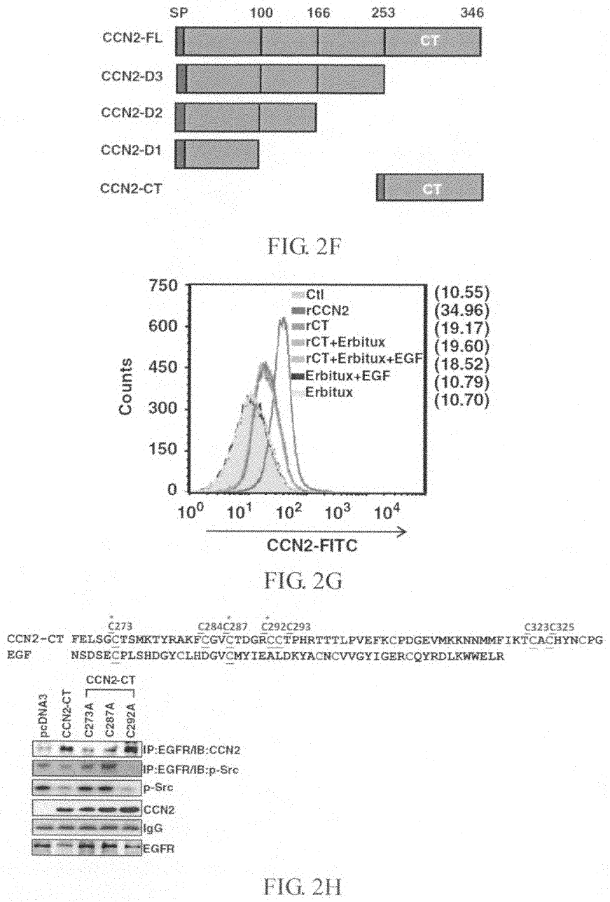

EXAMPLE 1: CCN2 binds to EGFR through the carboxyl-terminal cystine knot (CT) domain. This embodiment focuses on the association between CCN2 and EGFR. First, the expression of CCN2 in the CCN2 transfected lung adenocarcinoma cell lines, CL1-5 and A549, transduced with CCN2 and in the lung adenocarcinoma cell line, CL1-0, transduced with siCCN2 was observed by western blot analysis, the results shown in FIG. 2A. The results show that the expression of CCN2 in the lung adenocarcinoma cell line, A549, was enhanced, but the expression of CCN2 in lung adenocarcinoma cell lines, CL1-0, was suppressed.

Next, the membranous association between EGFR and CCN2 in CL1-5 and A549 lung adenocarcinoma cell lines was observed by Fluorescence-activated cell sorting (abbreviated as FACS), the results shown in FIG. 2B. The results show that in siRNA (control group), rCCN2 (Recombinant CCN2) (100 ng/mL), rCCN2(100 ng/mL)/siEGFR and rCCN2(100 ng/mL)/siRNA, rCCN2 enhanced the detection of membranous CCN2 and that depletion of EGFR in these cells abolished the CCN2 located on cell membrane. CCN2 has a concentration between 5 ng/mL and 200 ng/mL(data not shown). In this embodiment, the concentration thereof is about 100 ng/mL.

Next, the association strength between EGFR and CCN2 in the lung adenocarcinoma cell line, A549, was observed by immunoprecipitation and in vitro binding assay, the results shown in FIG. 2C and FIG. 2D. The results show that there is a physically association between CCN2 and EGFR (as shown on the right in the FIG. 2C). In addition, when added EGFR ligands EGF, do not block the physically association between CCN2 and EGFR (as shown on the left in the FIG. 2C, in this, the A549 lung adenocarcinoma cell lines were cotransfected with CCN2- and EGFR-expression plasmids (top left) or transfected with EGFR-expression plasmid and treated with rCCN2 (100 ng/mL) (bottom left), and then incubated with rEGF (20 ng/mL) for 24 h). In addition, when the depletion of tyrosine kinase receptor (abbreviated as TrkA) which associated with CCN2 did not alter the association strength between EGFR and CCN2 (as shown in FIG. 2D, in this, the scramble control or siTrkA (200 ng) was transfected into A549 lung adenocarcinoma cell lines).

Next, the effect of the EGFR monoclonal antibody, Erbitux, on the association of CCN2 and EGFR in the lung adenocarcinoma cell line, A549, was observed by fluorescence-activated cell sorting, the results shown in FIG. 2E. The results show that in rEGF (20 nM), Erbitux (1 μM), rEGF (20 nM)/IgG and rEGF (20 nM)/Erbitux (1μM), and in rCCN2 (100 nM), Erbitux (1 μM), rCCN2 (100 nM)/IgG and rCCN2 (100 nM)/Erbitux (1 μM), Erbitux does not affect the association between CCN2 and EGFR.

Next, the domain of CCN2 responsible for EGFR binding in the suspended lung adenocarcinoma cell line, A549, was observed by fluorescence-activated cell sorting and western blot analysis, the results shown in FIGS. 2F to 2H. The results show that in a series of CT-domain deletion constructs or CT-domain construct of CCN2(as shown in FIG. 2F), CT-domain of CCN2 has association relationship with EGFR, and the association was not affected by the EGF and Erbitux(as shown in FIG. 2G, rCCN2 (100 ng/mL), rCT (100 ng/mL) and Erbitux(1μM)). In addition, the CT domain of CCN2, which is highly conserved with EGF, was responsible for EGFR binding of CCN2. When cysteine (Abbreviated as C) residues C273A and C287A of the CT domain of CCN2 mutated to alanine (Abbreviated as A), significantly reduced the association between CCN2(mutated-type CCN2) and EGFR(as shown in FIG. 2H). In addition, when the CT domain of CCN2 unmutated (wild-type CCN2) or mutant occurred in cysteine residues C292A (mutated-type CCN2), it can reduce the association between EGFR and Src(as shown in FIG. 2H, in this use wild-type CCN2, mutated-type CCN2 and vector (pcDNA3) expression plasmids in the lung adenocarcinoma cell line, CL1-5, to observe).

In summary, CCN2 binds to EGFR through the cysteine residues C273A and C287A of the CT domain.

### EXAMPLE 2

### CCN2 Inhibits EGFR-Mediated Phosphorylation of c-Src and Extracellular Signal-Regulated Knase (ERK)

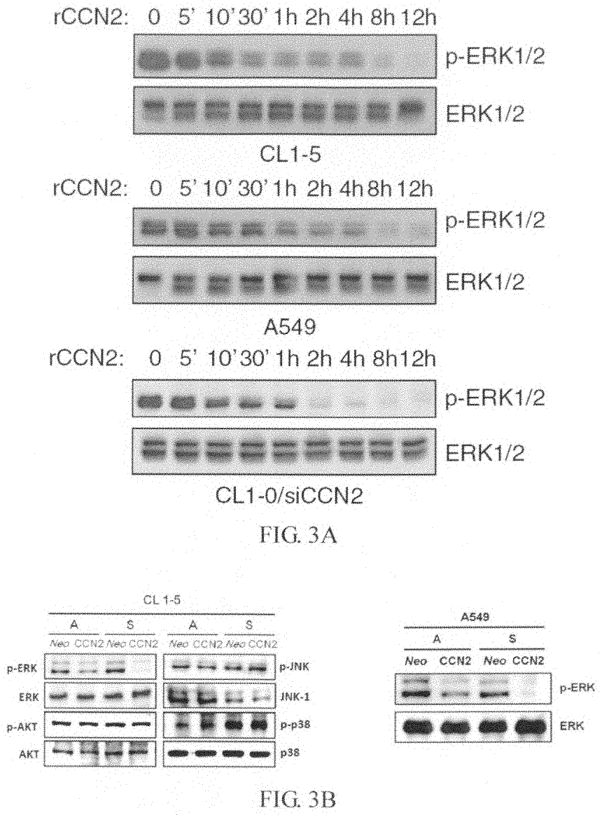

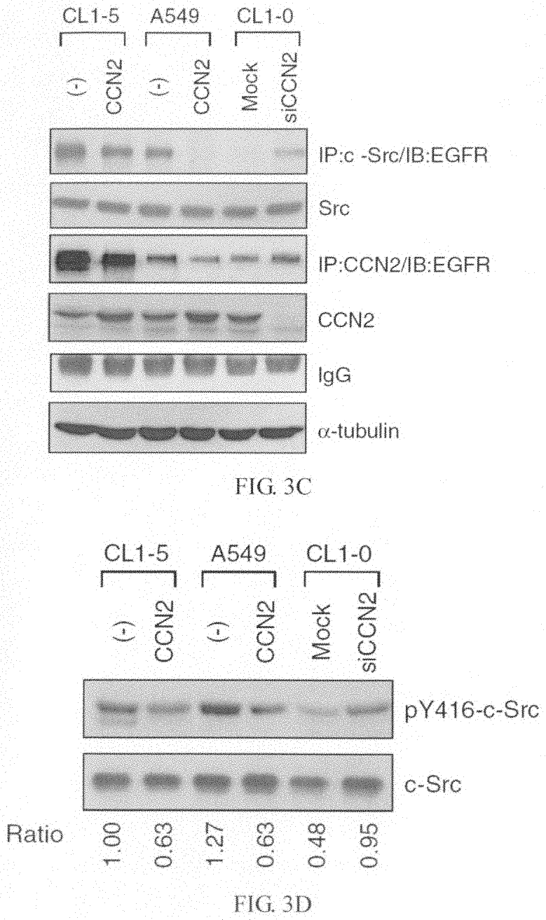

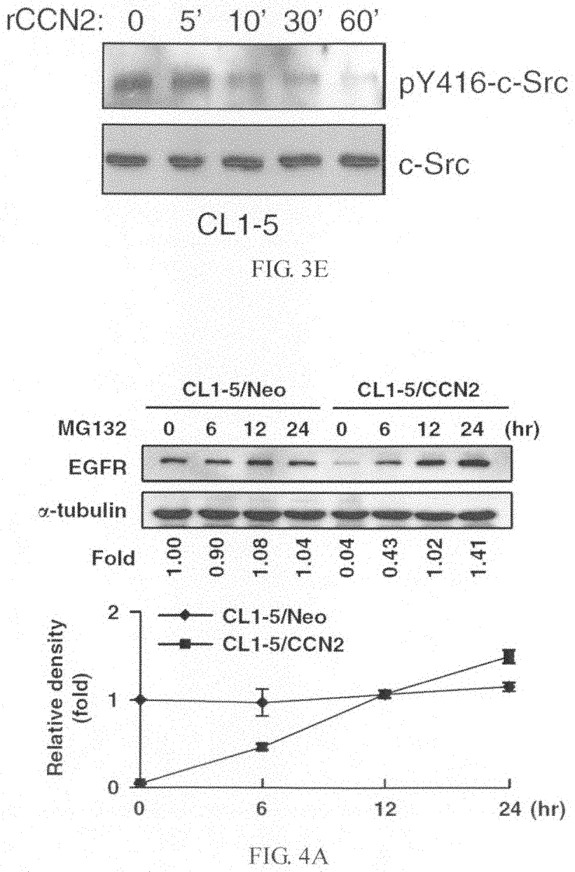

This embodiment focuses on the effect of CCN2 on the EGFR-induced signaling pathways. At first, the effect of exogenous CCN2(rCCN2) on the MAPK/AKT signaling pathways in the lung adenocarcinoma cell lines, CL1-5, A549 and CL1-0/siCCN2 was observed by western blot analysis, the results shown in FIG. 3A. The results show that in the lung adenocarcinoma cell lines, CL1-5, A549 and CL1-0/siCCN2rCCN2 cangradually reduce the phosphorylation of ERK (p-ERK1/2) with increasing time.

Next, the effect of CCN2 on the phosphorylation of ERK in attached and suspended lung adenocarcinoma cell lines, CL1-5 and A549 was observes by western blot analysis, the results shown in FIG. 3B (As shown on the left in FIG. 3B, CL1-5/CCN2 and Neo control cells were cultured in attached (A) or suspended (S) for 48 hr, and then the protein levels of p-ERK, total ERK, p-AKT, total AKT, p-JNK, total JNK1, p-p38 and total p38 was observed; as shown on the right of FIG. 3B, A549/CCN2 and Neo control cells were cultured in attached (A) or suspended (S) for 48 hr, and then the protein levels of p-ERK, total ERK was observed). The results show that in attached or suspended lung adenocarcinoma cell lines, CL1-5 and A549, CCN2 can significantly reduc the protein level of p-ERK, and inhibit the phosphorylation of ERK.

Next, the effect of CCN2 on the formation of EGFR/Src complex and the phosphorylation of Src in the lung adenocarcinoma cell lines, CL1-5, A549 and CL1-0/siCCN2, was observed by western blot analysis, the results shown in FIGS. 3C to 3E. The results show that in the lung adenocarcinoma cell lines, CL1-5 and A549, CCN2 can inhibit the formation of EGFR/c-Src complex(as shown in FIG. 3C) and promote the autophosphorylation of c-Src^{Y416} (as shown in FIG. 3D). In addition, in the lung adenocarcinoma cell line, CL1-5, rCCN2 can gradually inhibit the autophosphorylation of c-Src^{Y416}(as shown in FIG. 3E) with increasing time (0 minutes, 5 minutes, 10 minutes, 30 minutes, and 60 minutes). In addition, as shown in FIG. 2H of example 1, the CT-domain of CCN2 can also inhibit the phosphorylation of Src, and point mutation of cysteine residues C273A and C287A can activate the phosphorylation of Src and promote the formation of EGFR/Src complex.

In summary, the CT-domain of CCN2 may prevent the formation of EGFR/Src complex and inhibit the phosphorylation of c-Src.

### EXAMPLE 3

### CCN2 Induces Ubiquitination-Dependent Degradation of EGFR By Disrupting the p-pix /c-casitas B-lineage Lymphoma (c-Cbl) Complex

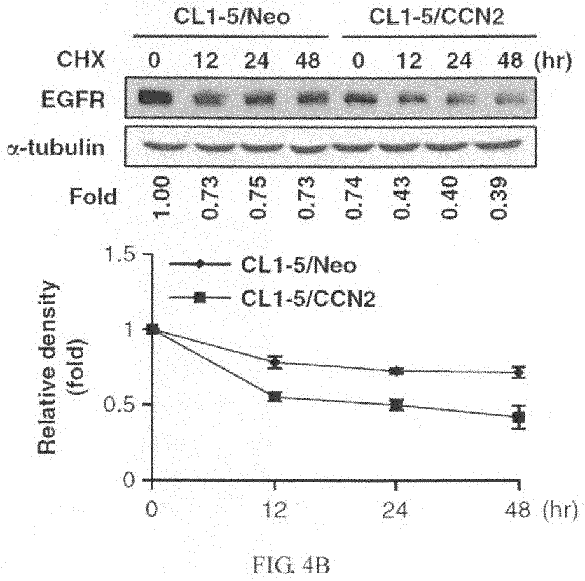

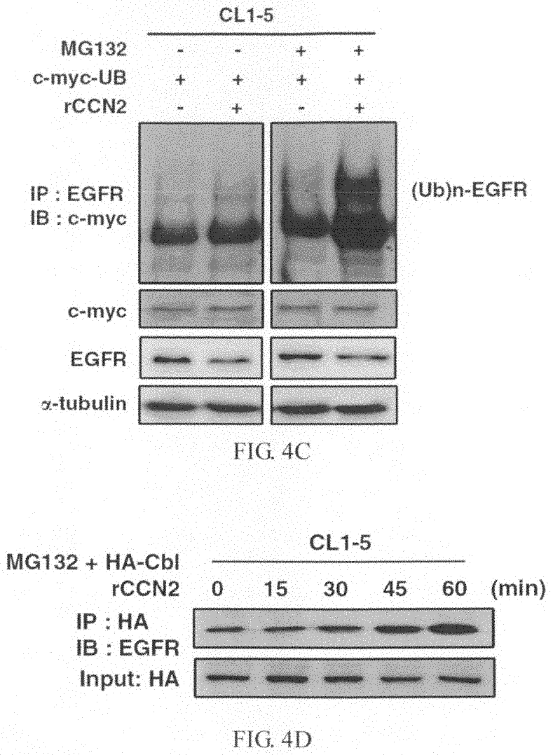

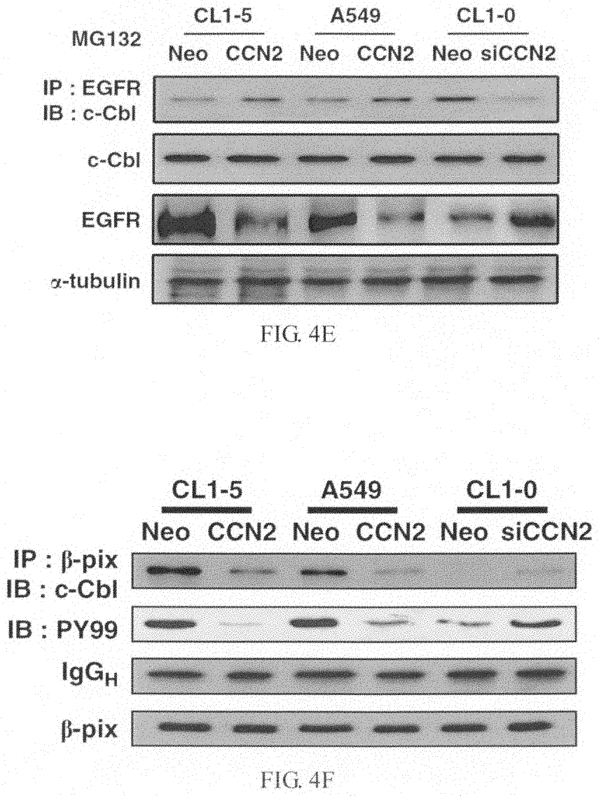

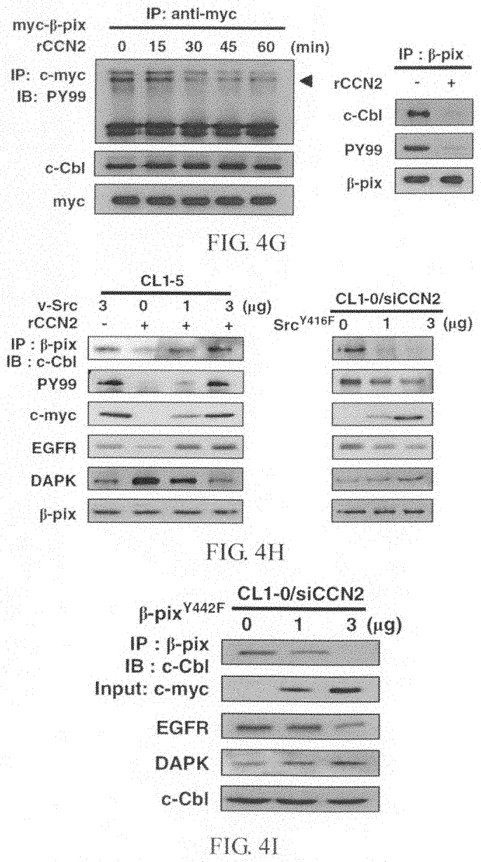

This embodiment focuses on whether CCN2 interacts with EGFR. At first, the effect of MG132 and CCN2 on the degradation of EGFR in the lung adenocarcinoma cell line, CL1-5, and the effect of cycloheximide and CCN2 on the degradation of EGFR in the lung adenocarcinoma cell line, CL1-5,were observed by western blot analysis, the results shown in FIGS. 4A and 4B. The results show that MG132 can gradually suppress the CCN2-induced EGFR degradation and gradually enhance the protein signal intensity(Fold) of EGFR (enhanced from 0.04 to 1.41)(as shown in FIG. 4A) with increasing time (0 hours, 6 hours, 12 hours, 24 hours). On the other hand, cycloheximide can gradually promote CCN2-induced EGFR degradation and attenuated the protein signal intensity(Fold) of EGFR (attenuated from 0.74 to 0.39)(as shown in FIG. 4B) with increasing time (0 hours, 12 hours, 24 hours, 48 hours). Thus, CCN2 can destabilize EGFR and enhance the degradation of EGFR.

Next, the effect of rCCN2 on the EGFR ubiquitination degree and the formation of EGFR/c-Cbl complex in the lung adenocarcinoma cell line, CL1-5, was observed by western blot analysis, the results shown in FIGS. 4C to 4E. The results show that rCCN2 can increase the EGFR ubiquitination degree (as shown in FIG. 4C, cell lysates of the lung adenocarcinoma cell line, CL1-5, transiently transfected with c-myc-ubiquitin-expressing plasm ids were immunoprecipitated with anti-EGFR and immunoblotted with monoclonal mouse anti-c-myc-ubiquitin), and can gradually increase the formation of EGFR/c-Cbl complex(as shown in FIG. 4D, cell lysates of the lung adenocarcinoma cell line, CL1-5, transiently transfected HA-tag c-Cbl-expressing plasmids with MG132, were immunoprecipitated with anti-HA and immunoblotted with anti-EGFR) with increasing time (0 minutes, 15 minutes, 30 minutes, 45 minutes, 60 minutes). In addition, in the lung adenocarcinoma cell lines, CL1-5 and A549, the formation of EGFR/c-Cbl complex was increased, but in the lung adenocarcinoma cell line, CL1-0/siCCN2, the formation of EGFR/c-Cbl complex was decreased(as shown in FIG. 4E, cell lysates of the lung adenocarcinoma cell line, CL1-5, were immunoprecipitated with anti-EGFR and immunoblotted with anti-c-Cbl).

Next, the effect of CCN2 on the formation of β-pix/c-Cbl complex and the phosphorylation of β-pix in the lung adenocarcinoma cell lines, CL1-5, A549 and CL1-0/siCCN2, was observed by western blot analysis, the results shown in FIG. 4F(cell lysates of the lung adenocarcinoma cell lines, CL1-5, A549 and CL1-0/siCCN2, were immunoprecipitated with anti-human b-pix antibody and anti-c-Cbl, and immunoblotted with anti-PY99 and anti-b-pix). The results show that in the lung adenocarcinoma cell lines, CL1-5 and A549, 13-pix/c-Cbl complex was reduced and the phosphorylation of β-pix was decreased, but in CL1-0/siCCN2 lung adenocarcinoma cell lines, the formation of β-pix/c-Cbl complex was increased, and the phosphorylation of β-pix was enhanced.

Next, the effect of exogenous CCN2 (rCCN2) on the formation of β-pix/c-Cbl complex and the phosphorylation of β-pix in the lung adenocarcinoma cell line, CL1-5, was observed by western blot analysis, the results shown in FIG. 4G. The results show that rCCN2 can gradually reduce the formation of β-pixie-Chi complex(as shown on the left in the FIG. 4G, cell lysates of the lung adenocarcinoma cell line, CL1-5, were transiently transfected with c-myc-tagged b-pix-expressing plasmids, immunoprecipitated with anti-myc, and immunoblotted with PY99), and reduces β-pix phosphorylation(as shown on the right in the FIG. 4G, in this, after 30 min, cell lysates of the lung adenocarcinoma cell line, CL1-5, were immunoprecipitated with anti-β-pix, and immunoblotted with c-Cbl and PY99) with increasing time (0 minutes, 15 minutes, 30 minutes, 45 minutes, 60 minutes),.

Next, the effect of v-Src, SrC^{Y416F} mutant, β-piX^{Y422F} mutant and rCCN2 on the formation of β-pix/c-Cbl complex, the phosphorylation of β-pix and EGFR degradation in the lung adenocarcinoma cell lines, CL1-5 and CL1-0/siCCN2, was observed by western blot analysis, the results shown in FIGS. 4H and 41. The results show that when the dose of v-Src gradually increased (0 μg, 1 μg, 3 μg) in the lung adenocarcinoma cell line, CL1-5, rCCN2 could promote the formation off β-pix/c -Chl complex and the phosphorylation of β-pix to inhibit EGFR degradation(as shown on the left in the FIG. 4H, CL1-5 transiently transfected v-Src-expressing plasmids with rCCN2 treatment for 15 min). In contrast, when the number of SrC^{Y416F} mutants in the lung adenocarcinoma cell line, CL1-0/siCCN2, was increased, the formation of β-pix/c-Cbl complex and the phosphorylation of β-pix were inhibited to enhance EGFR degradation(as shown on the right in the FIG. 4H, CL1-0/siCCN2 clone transiently transfected point mutation of SrcY416F-expressing plasmids for 48 h. Cell lysates were immunoprecipitated with anti-β-pix and immunoblotted with anti-c-Cbl and PY99). In addition, when the number of β-pix^{Y422F} mutant in CL1-0/siCCN2 lung adenocarcinoma cell lines was increased, the formation of β-pix/c-Cbl complex was inhibited to enhance EGFR degradation and DAPK expression(as shown in FIG. 4I, in this, CL1-0/siCCN2 transiently transfected point mutation of β-pix^{Y422F}-expressing plasmids. Cell lysates were immunoprecipitated with anti-β-pix and immunoblotted with anti-c-Cbl).

In summary, CCN2 can disrupt the formation of β-pix/c-Cbl complex to induce EGFR degradation by inhibiting Src-mediated β-pix phosphorylation.

### EXAMPLE 4

### CCN2 Induces Anoikis in Lung Adenocarcinoma Cells

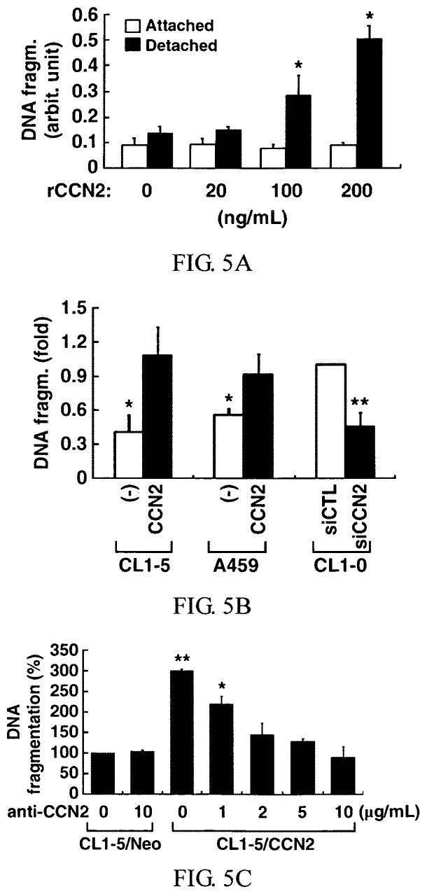

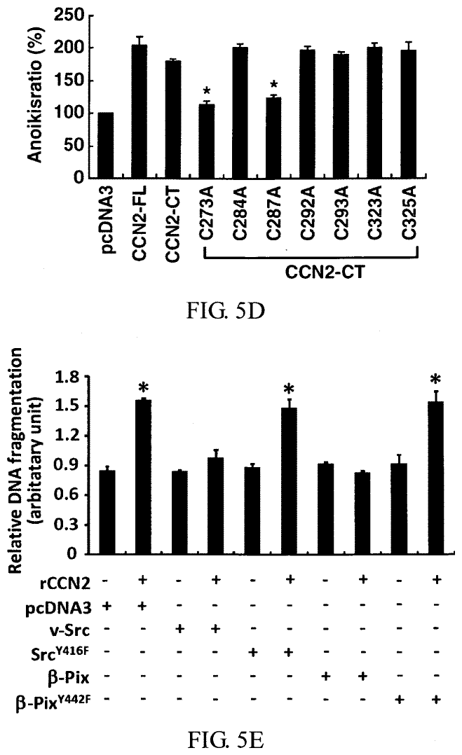

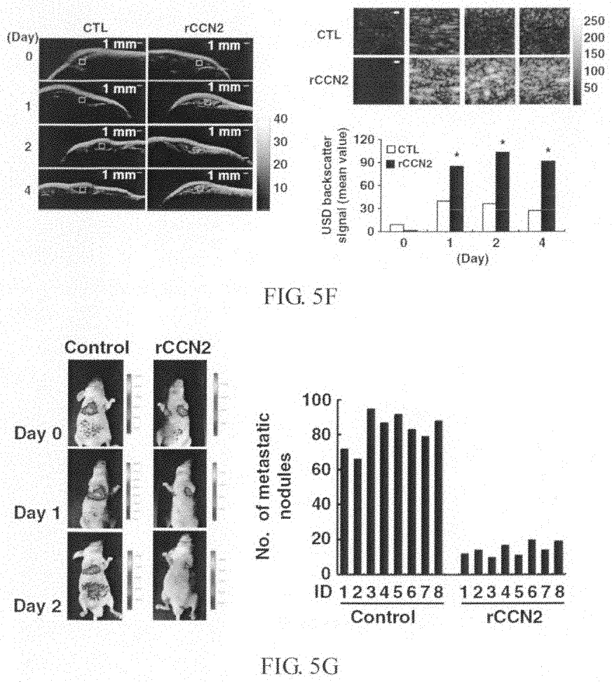

This embodiment focuses on whether CCN2 can induce anoikis in lung adenocarcinoma cells in vitro and in vivo. At first, CCN2 induces anoikis which is induced by CCN2 in lung adenocarcinoma cells in vitro was observed by anoikis assay, the results shown in FIGS. 5A to 5C. The results show that when the lung adenocarcinoma cell line, CL1-5, was attached, DNA fragmentation which was induced by rCCN2 with various concentrations (0 ng/mL, 20 ng/mL, 100 ng/mL, 200 ng/mL) has no obvious difference. However, when the lung adenocarcinoma cell line, CL1-5, wassuspended, DNA fragmentation which is induced by rCCN2 with higher concentration was increased (as shown in FIG. 5A, data was means±S.E. (n=6 per group). *P<0.05). In addition, when thelung adenocarcinoma cell lines, CL1-5 and A549, were suspended, DNA fragmentation was increased, but when the lung adenocarcinoma cell line, CL1-0/siCCN2, was suspended, DNA fragmentation was decreased(as shown in FIG. 5B, in this, CL1-5, A549, and CL1-0 cells were transfected with either CCN2 or siCCN2 treatment for 24 h). In addition, when the lung adenocarcinoma cell line, CL1-5, were suspended, anti-CCN2 (0 μg/mL, 1 μg/mL, 2 μg/mL, 5 m/mL, 10 μg/mL) with the higher concentration can reduce the DNA fragmentation more significantly(as shown in FIG. 5C, in this, data was means±S.E. (n=4 per group). *P<0.05 and **P<0.01).

Next, the effect of different CCN2 mutants(also be referred to as CCN2 variants), Src and β-pix effect on anoikis in the suspended lung adenocarcinoma cell line, CL1-5, was observed by anoikis assay, the results shown in FIGS. 5D and 5E. The results show that when the mutations of cysteine residues C273A and C287A of the CT domain of CCN2 can inhibit anoikis(as shown in FIG. 5D, in this, CCN2 variants were transiently transfected and suspended for 24 h). In addition, Src^{Y416F} mutant and β-pix^{Y422F} mutant can promote anoikis, but the v-Src and β-pix can suppress anoikis(as shown in FIG. 5E, in this, the lung adenocarcinoma cell line, CL1-5, were transient-transfected with v-Src, point mutation SrC^{Y416F} construct, β-pix, point mutation β-pix^{Y442F} construct, or control vector pcDNA3 for 48 hr and suspended to induce anoikis. Data was means±S.E. (n=6 per group). *P<0.05).

In summary, CCN2 induced anoikist can be enhanced by inhibiting Src activation and β-pix phosphorylation.

Next, the effect of rCCN2 on anoikis in the lung adenocarcinoma cell line, CL1-5, (in vivo) was observed by high-frequency ultrasonic imaging, the results shown in FIG. 5F(top right indicated as the amplification view of the yellow squares). In this embodiment, the lung adenocarcinoma cell line, CL1-5, was injected into the s.c. region of nude mice and the mice was treated i.p. either with rCCN2 (1 mg/kg per day) or a control solution (citrate buffer), and anoikis in the lung adenocarcinoma cell line, CL1-5,(in vivo) was observed by high-frequency ultrasonic imaging. The results show that the nude mice which were injected with rCCN2 clearly appeared anoikis.

Next, after the lung adenocarcinoma cell line, CL1-5,(in vivo) i.v. injected into nude mice treated with or without CCN2 (1 mg/kg per day), the change of the metastasis in the lung adenocarcinoma cell line, CL1-5,(in vivo) was observed by celltracker probe (Invitrogen Corporation, Carlsbad, Calif., USA), the results shown in FIG. 5G. The results show that the signals in lungs were found to be much weaker in CCN2-treated nude mice (as shown on the left in the FIG. 5G). In addition, the number of metastatic lung nodules was significantly reduced in CCN2-treated nude mice (as shown on the right in the FIG. 5G).

In summary, CCN2 can induce anoikis in vitro and in vivo and inhibit the metastasis of lung adenocarcinoma cells.

### EXAMPLE 5

### DAPK-Dependent Anoikis is Critical in CCN2-Mediated Metastatic Suppression

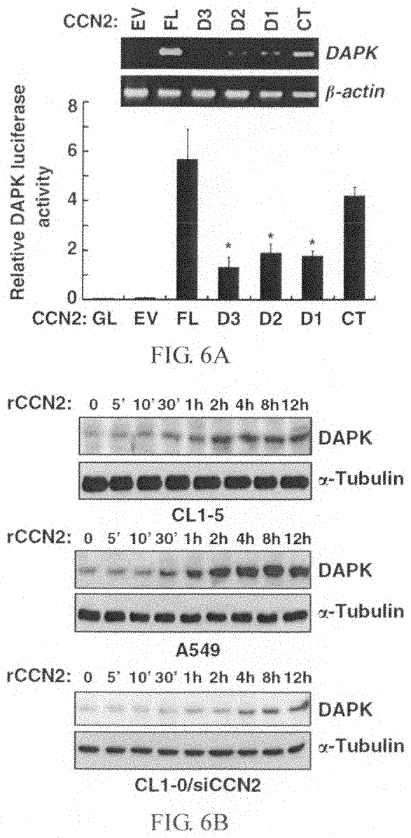

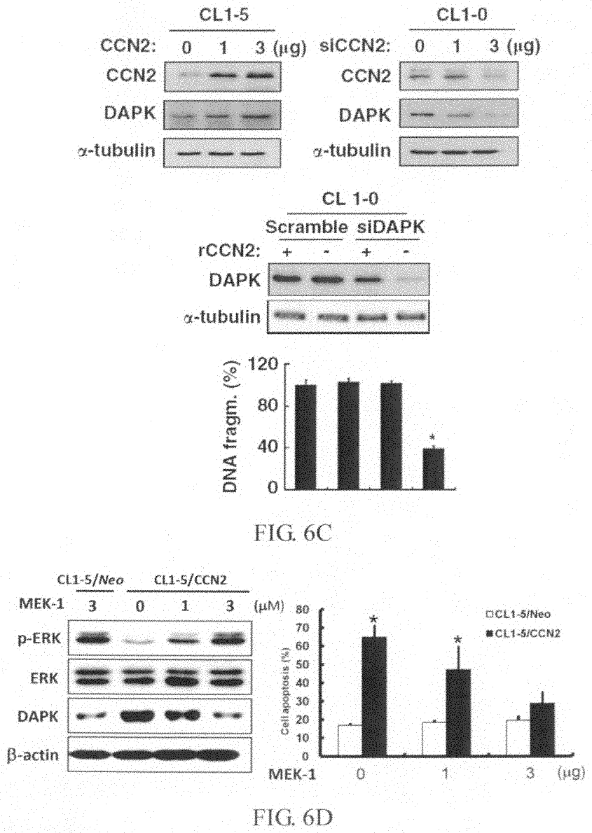

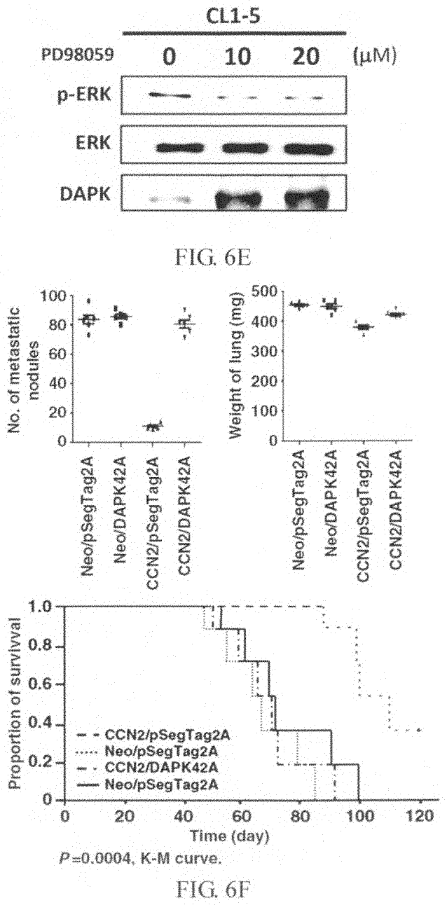

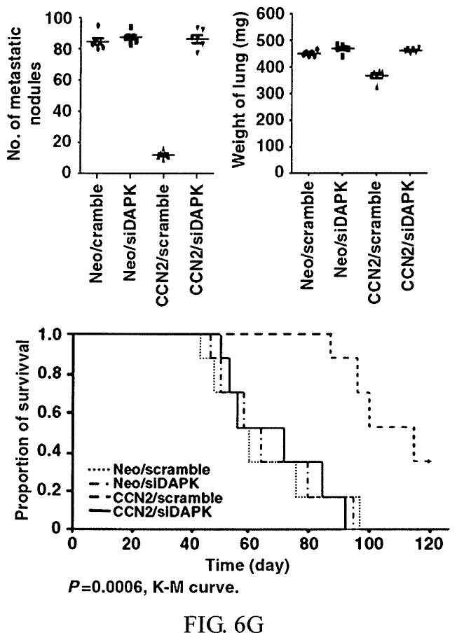

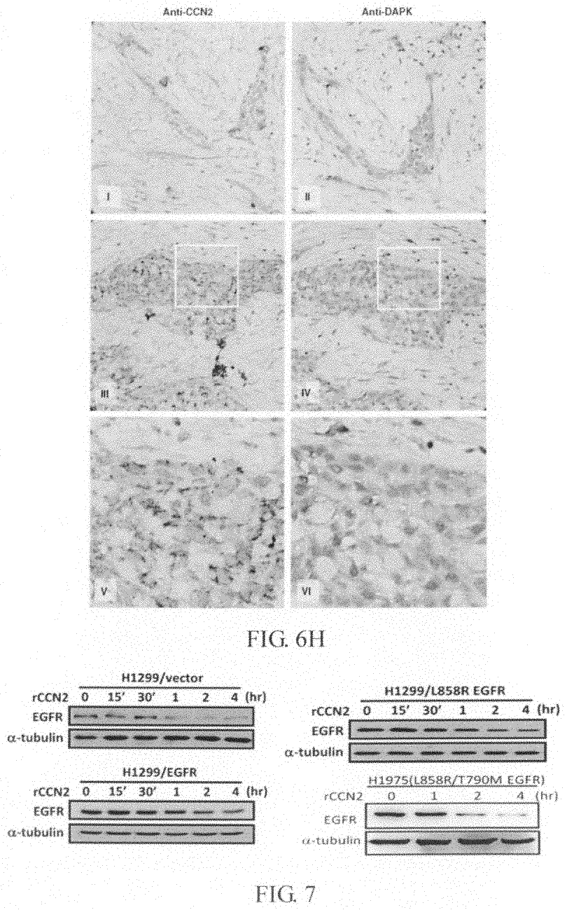

This embodiment focuses on the downstream signaling in CCN2-induced anoikis. At first, the effect of DAPK which can up-regulate CCN2 played on CCN2 induced anoikis and metastasis inhibition of lung adenocarcinoma cell lineswas observed by RT-PCR, dual luciferase reporter assay and western blot analysis, the results shown in FIGS. 6A to 6C. The results show that in the lung adenocarcinoma cell line, CL1-5, CCN2 (FL and CT) with a CT domain can promote the activity of DAPK mRNA (as shown in FIG. 6A, the lung adenocarcinoma cell line, CL1-5, was transfected for 48 h). In addition, in the lung adenocarcinoma cell lines, CL1-5, A549 and CL1-0/siCCN2, rCCN2 can promote DAPK expression, and DAPK expression can also be increased with increasing time (0 minutes, 5 minutes, 10 minutes, 30 minutes, 1 hour, 2 hours, 4 hours, 8 hours, 12 hours) (as shown in FIG. 6B). In addition, in the lung adenocarcinoma cell line, CL1-5, an exogenous CNN2 (rCCN2) can promote DAPK expression, and DAPK expression also be increased with increasing dose (0 μm, 1μg, 3 μg), (as shown on the top left in the FIG. 6C). In addition, in the lung adenocarcinoma cell line, CL1-0/siCCN2, DAPK expression is suppressed, and DAPK expression also be suppressed with increasing dose (0 μg, 1μg, 3μg), (as shown on the top right in the FIG. 6C). Therefore, CCN2 induced DAPK expression in time- and dose-dependent manners. In addition, in the lung adenocarcinoma cell line, CL1-0/siCCN2, when DAPK expression was significantly decreased due to siRNA, rCCN2 induced DNA fragmentation wasalso decreased(as shown on the bottom in the FIG. 6C, apoptotic cells in suspension were detected at 24 h).

Next, the signaling pathway of DAPK expression was observed by western blot analysis, the results shown in FIGS. 6D and 6E. The results show that in the lung adenocarcinoma cell line, CL1-5, MEK-1 which can up-regulate MAPK/ERK pathway, with increasing concentrations (0 μM, 1 μM, 3 μM), can gradually increase the p-ERK expression and decrease' DAPK expression, resulting in anoikis resistance (as shown in FIG. 6D, transient introduced MEK-1 expressing vector into CL1-5/CCN2 and CL1-5Neo cells for 48 hr). In addition, in the lung adenocarcinoma cell line, CL1-5, an ERK inhibitor PD98059 with increasing concentrations (0 μM, 10 μM, 20 μM), can gradually inhibit p-ERK and gradually increase the DAPK expression(as shown in FIG. 6E).

In summary, CCN2 promotes DAPK-dependent anoikis by inhibiting ERK/MAPK signaling pathway.

Next, whether CCN2-induced metastatic suppression in the suspended lung adenocarcinoma cell lines through the DAPK-induced anoikis was observed by Kaplan-Meier survival plots, the results shown in FIGS. 6F and 6G. In this embodiment, the mice were injected i.v. with the lung adenocarcinoma cell line, CL1-5. The results show that when Neo/pSecTag2A (control group), Neo/DAPK42A, CCN2/pSecTag2A and CCN2/DAPK42A are used, the mice injected with CCN2/pSecTag2A can significantly reduce the number of metastatic lung nodules and weight of lung; the mice injected with CCN2/DAPK42A doesn't show an obviously reduced number of metastatic lung nodules and weight of lung, but it can significantly reduce survival period (as shown in FIG. 6F, in this, p-value was determined by a two-sided log-rank test, p=0.0004). In addition, when Neo/cramble, Neo/siDAPK, CCN2/scramble and CCN2/siDAPK are used, the mice injected with CCN2/scramble can significantly reduce number of metastatic lung nodules and weight of lung; the mice injected with CCN2/siDAPK although doesn't show an obviously reduced number of metastatic lung nodules and weight of lung, but it can significantly reduce survival period (as shown in FIG. 6G, in this, p-value was determined by a two-sided log-rank test, p=0.0006).

Next, the effect of CCN2 and DAPK on the lung adenocarcinoma patients was observed by immunohistochemistry, the results shown in FIG. 6H. The results show that in 27 lung adenocarcinoma patients, a positive correlation between DAPK and CCN2 protein levels. These findings confirmed that a CCN2-DAPK signal axis occurred in human lung adenocarcinoma cell lines.

### EXAMPLE 6

### CCN2 Can Inhibit Wild/Mutated-Type EGFR Expression

This embodiment shows the effect of rCCN2 on the expression of wild-type and mutated-type EGFR. First, the effect of rCCN2 on the wild-type EGFR 1-11299 lung adenocarcinoma cell line, the mutated-type EGFR H1299 lung adenocarcinoma cell line, and the mutated-type EGFR H1975 lung adenocarcinoma cell line was observed by western blot analysis, the results shown in FIG. 7(Cell lysates of H1299 transfectants or H1975 cells were immunoblotted with anti-EGFR). The results show that in the 1-11299 lung adenocarcinoma cell lines (wild-type EGFR), the H1299 lung adenocarcinoma cell lines (single mutation point, L858R) and the H1975 lung adenocarcinoma cell lines(two mutation points, L858R and T790M), rCCN2 can inhibit the expression of EGFR more significantlywith increasing treatment time (0 minutes, 15 minutes, 30 minutes, 1 hour, 2 hours, 4 hours, H1975 lung adenocarcinoma cell lines are only 0 hours, 1 hour, 2 hours, 4 hours).

### EXAMPLE 7

### CCN2 Can Increase the Sensitivity of Chemotherapy and EGFR Targeted Therapy in Lung Adenocarcinoma Cell Lines

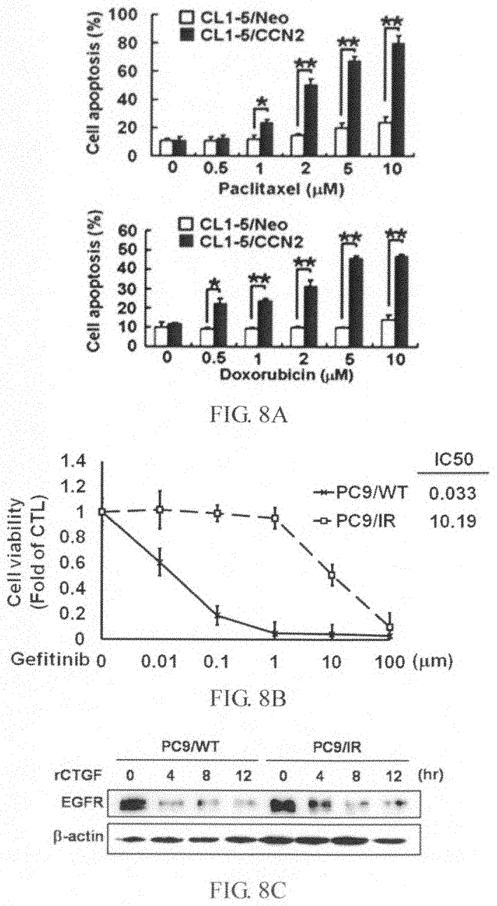

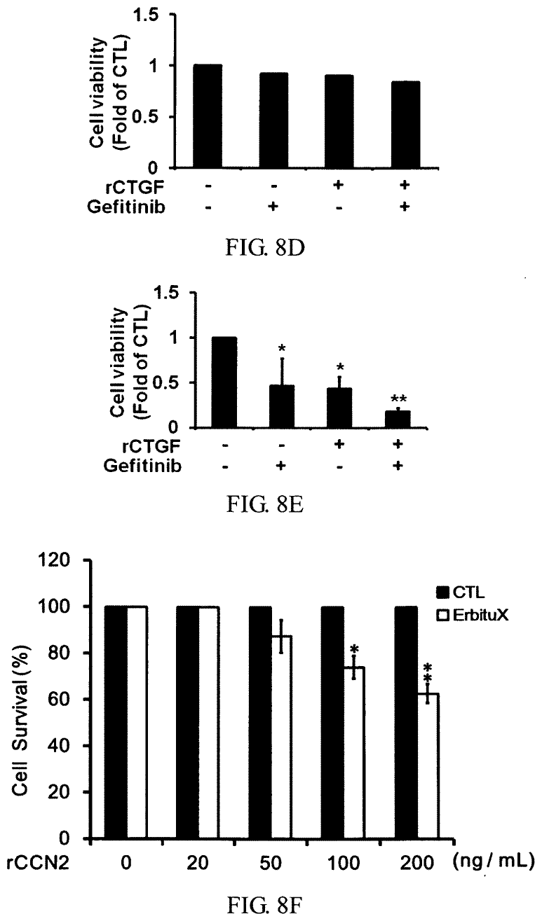

This embodiment shows the effect of CCN2 on chemotherapy and EGFR targeted therapy. First, the effect of CCN2 on the suspended lung adenocarcinoma cell line, CL1-5, treated with chemotherapy was observed by MTT Assay, the results shown in FIG. 8A (Cells were treated for 24 hrs, *P<0.05, **P<0.01). The results show that when the dose of chemotherapy drugs, paclitaxel and doxorubicin, increasese (0 μM, 0.5 μM, 1 μM, 2 μM, 5 μM, 10 μM), the CCN2 transfected lung adenocarcinoma cell line, CL1-5, can increase cell apoptosis more significantly. In this embodiment, paclitaxel treatment with a concentration between 1 μM and 10 μM is perferred, and 10 μM is most preferable; doxorubicin treatment with a concentration between 0.5 μM and 10 μM is perferred, and 5 μM is most preferable.

Next, the effect of EGFR tyrosine kinase inhibitor, gefitinib, on PC9/WT and PC9/IR lung adenocarcinoma cell lines is observed by MTT Assay and western blot analysis, and the results are shown in FIG. 8B (In FIG. 8B, cells were treated for 48 hrs). The results show that in different treatment dosage (0 μM, 0.01 μM, 0.1 μM, 1 μM, 10 μM, 100 μM), IC50 of suspended PC9/WT lung adenocarcinoma cell lines is 0.033 μM, while IC50 of suspended PC9/IR drug resistant lung adenocarcinoma cell lines is 10.19 μM. Therefore, the sensitivity of the suspended PC9/IR drug resistant lung adenocarcinoma cell lines to gefitinib are lower.

Next, the effect of rCCN2 on attached PC9/WT lung adenocarcinoma cell lines and attached PC9/IR drug resistant lung adenocarcinoma cell lines is observed by MTT Assay and western blot analysis, and the results are shown in FIG. 8C. The results show that under 100 ng/mL rCCN2 treatment, when increasing the treatment time (0 hours, 4 hours, 8 hours, 12 hours), the expression of EGFR in attached PC9/WT lung adenocarcinoma cell lines and attached PC9/IR drug resistant lung adenocarcinoma cell lines will be suppressed more significantly.

Next, the effect of rCCN2 and EGFR tyrosine kinase inhibitor, gefitinib, on the PC9/IR drug resistant lung adenocarcinoma cell lines is show by MTT Assay and western blot analysis, and the results are shown in FIGS. 8D and FIG. 8E. The results show that when PC9/IR drug resistant lung adenocarcinoma cell lines are in attached condition, under 100 ng/mL rCCN2 and 40 μM EGFR tyrosine kinase inhibitor gefitinib cotreatment, cell viability cannot be significantly reduced (as shown in FIG. 8D, Anoikis assay were measured after 24 hours, **P<0.01). But when PC9/IR drug resistant lung adenocarcinoma cell lines are in suspended condition, under 100 ng/mL rCCN2 and 40 μM EGFR tyrosine kinase inhibitor gefitinib cotreatment, cell viability can be significantly reduced (as shown in FIG. 8E, Anoikis assay were measured after 24 hours, *P<0.05, **P<0.01).

Next, the effect of rCCN2 and EGFR monoclonal antibody, cetuximab (brand name Erbitux), on the CL1-5 lung adenocarcinoma cell lines is observed by MTT Assay and western blot analysis, and the results are shown in FIG. 8F. The results show that when using different concentrations rCCN2 alone treatment, increases with concentrations (0 ng/mL, 20 ng/mL, 50 ng/mL, 100 ng/mL, 200 ng/mL), cell survival of attached CL1-5 lung adenocarcinoma cell lines was not significantly reduced, but when in cotreatment with 2 mM EGFR monoclonal antibody cetuximab (brand name Erbitux), cell survival of attached CL1-5 lung adenocarcinoma cell lines was significantly reduced.

What is claimed is:

1. A combination for inhibiting the cancer cells, comprising a pharmaceutically effective dose of CCN2 or a CCN2 functional fragment; and
a pharmaceutically effective dose of a cancer drug.

2. The combination according to claim 1, wherein the CCN2 functional fragment comprises a CT domain.

3. The combination according to claim 1, wherein the CCN2 functional fragment comprises cysteines in positions 273 and 287, counted from the N-terminus.

4. The combination according to claim 1, wherein CCN2 or the CCN2 functional fragment has a concentration between 5 ng/mL and 200 ng/mL.

5. The combination according to claim 1, wherein the cancer drug is a cancer chemotherapy drug.

6. The combination according to claim 5, wherein the cancer chemotherapy drug is paclitaxel or doxorubicin.

7. The combination according to claim 6, wherein paclitaxel has a concentration between 1 μM and 10 μM.

8. The combination according to claim 6, wherein doxorubicin has a concentration between 0.5 μM and 10 μM.

9. The combination according to claim 1, wherein the cancer drug is an EGFR targeted drug.

10. The combination according to claim 9, wherein the EGFR targeted drug is an EGFR tyrosine kinase inhibitor or an EGFR monoclonal antibody.

11. The combination according to claim 10, wherein the EGFR tyrosine kinase inhibitor is gefitinib.

12. The combination according to claim 10, wherein the EGFR monoclonal antibody is cetuximab.

13. The combination according to claim 1, wherein the cancer cells are non-small cell lung cancer cells, large intestine cancer cells, colorectal cancer cells, breast cancer cells, pancreatic cancer, ovarian cancer, AIDS-related Kaposi's sarcoma cells, head and neck cancer, squamous cell carcinoma, colon cancer cells, skin cancer cells, prostate cancer cells, cervical cancer cells, gastric cancer cells, esophageal cancer cells, brain cancer cells or bladder cancer cells.

14. A method for inhibiting the cancer cells, comprising administrating to the cancer cells a pharmaceutically effective dose of CCN2 or a CCN2 functional fragment and a pharmaceutically effective dose of a cancer drug.

15. The method according to claim 14, wherein the CCN2 functional fragment comprises a CT domain.

16. The method according to claim 14, wherein the CCN2 functional fragment comprises cysteines in positions 273 and 287, counted from the N-terminus.

17. The method according to claim 14, wherein the cancer drug is a cancer chemotherapy drug.

18. The method according to claim 17, wherein the cancer chemotherapy drug is paclitaxel or doxorubicin.

19. The method according to claim 14, wherein the cancer drug is an EGFR targeted drug.

20. The method according to claim 19, wherein the EGFR targeted drug is an EGFR tyrosine kinase inhibitor or an EGFR monoclonal antibody.

21. The method according to claim 20, wherein the EGFR tyrosine kinase inhibitor is gefitinib.

22. The method according to claim 20, wherein the EGFR monoclonal antibody is cetuximab.

23. The method according to claim 14, wherein the cancer cells are non-small cell lung cancer cells, large intestine cancer cells, cancer cells, breast cancer cells, pancreatic cancer, ovarian cancer, AIDS-related Kaposi's sarcoma cells, head and neck cancer, squamous cell carcinoma, colon cancer cells, skin cancer cells, prostate cancer cells, cervical cancer cells, gastric cancer cells, esophageal cancer cells, brain cancer cells or bladder cancer cells.


SEQUENCE LISTING (1 sequence)

SEQ ID NO: 1 - protein, 349 aa, Homo sapiens
  ref <301>: AUTHORS: Chang, C C, Yang, M H, Lin, B R, Chen, S T, Pan, S H, Hsiao, M, Lai, T C, Lin, S K, Jeng, Y M, Chu, C Y, Chen, R H, Yang, P C, Chin, Y E, Kuo, M L
  ref <302>: TITLE: CCN2 inhibits lung cancer metastasis through promoting DAPK-dependent anoikis and inducing EGFR degradation
  ref <303>: JOURNAL: Cell Death and Differentiation
  ref <304>: VOLUME: 20
  ref <306>: PAGES: 443-455
  ref <307>: DATE: 2012-11-23
MTAASMGPVRVAFVVLLALCSRPAVGQNCSGPCRCPDEPAPRCPAGVSLVLDGCGCCRVCAKQLGELCTERDPCDPHKGLFCHFGSPANRKIGVCTAKDGAPCIFGGTVYRSGESFQSSCKYQCTCLDGAVGCMPLCSMDVRLPSPDCPFPRRVKLPGKCCEEWVCDEPKDQTVVGPALAAYRLEDTFGPDPTMIRANCLVQTTEWSACSKTCGMGISTRVTNDNASCRLEKQSRLCMVRPCEADLEENIKKGKKCIRTPKISKPIKFELSGCTSMKTYRAKFCGVCTDGRCCTPHRTTTLPVEFKCPDGEVMKKNMMFIKTCACHYNCPGDNDIFESLYYRKMYGDMA